(19) United States
(12) Patent Application Publication
(10) Pub. No.: US 2007/0141654 A1
(43) Pub. Date: Jun. 21, 2007

(54) ELISA KIT FOR DIAGNOSING INFECTIOUS BURSAL DISEASE (IBD) AND METHOD USING THE SAME

(75) Inventors: Min-Ying Wang, Taichung (TW); Hung-Ling Hu, Ping-Tung County (TW)

Correspondence Address: PAI PATENT & TRADEMARK LAW FIRM, 1001 FOURTH AVENUE, SUITE 3200, SEATTLE, WA, 98154, US

(73) Assignee: BUREAU OF ANIMAL AND PLANT HEALTH INSPECTION AND QUARANTINE, Taipei (TW)

(21) Appl. No.: 11/306,153

(22) Filed: Dec. 17, 2005

(51) Int. Cl. G01N 33/00

(52) U.S. Cl. 435/7.92

(57) ABSTRACT

The present invention relates to an ELISA kit for diagnosing infectious bursal disease (IBD). In particular, the present invention provides an indirect ELISA kit comprising recombinant VP2H or VP3 as the antigen for detecting anti-IBDV antibodies in serum samples. The present invention relates to a method for diagnosing infectious bursal disease (IBD) by using the ELISA kit. The present invention also provides a method for preparing the recombinant VP3 protein with high recovery yield in E. coli expression system.

### BACKGROUND OF THE INVENTION

1. Field of the Invention

The present invention relates to an ELISA kit for diagnosing infectious bursal disease (IBD). In particular, the present invention provides an indirect ELISA kit comprising recombinant VP2H or VP3 as the antigen for detecting anti-IBDV antibodies in serum samples. The present invention relates to a method for diagnosing infectious bursal disease (IBD) by using the ELISA kit. The present invention also provides a method for preparing the recombinant VP3 protein with high recovery yield in E. coli expression system.

2. Description of the Related Prior Art

Infectious bursal disease (IBD) is a highly contagious immuno-suppressive disease in young chickens (between 3 and 6 weeks of age), in which Infectious bursal disease virus (IBDV) is the causative agent. The immunosuppression results from a depletion of B lymphocytes, and consequently led to death for the susceptibility to other pathogens (Fahey et al., J. Gen. Virol. 1989, 70, 1473-1481). In the route of contagion, IBD is spread rapidly by contacting contaminated water and food. It mainly causes symptoms of diarrhea, asthenia, anorexia, and weight loss (Cosgrove et al., Avian Dis., 1962, 6, 385-389). Since the burst of IBD in 1957 in USA, IBD has been spread over the chicken-rearing countries. As described in previous IBDV studies, the infection of chickens by IBDV could cause serious economic losses in the poultry industry worldwide, either by causing a high-mortality acute condition or by leading to immunosuppression in young chickens. Therefore, for controlling the IBDV infection in chicken, it is urgent to develop a rapid and specific IBD diagnosing method addition to the studies in subunit vaccine such as recombinant VP2 particles (see, for example, Wang et al., Proc. Biochem. 2000, 35, 877-884).

In the past, the diagnostic of IBD was carried out by examinations of specific clinical symptoms and pathologies. The clinical judgment of IBD may be confused by the mixed infection with other pathogens, and some IBDV-infected chickens express no significant clinical symptoms. So far, polymerase chain reaction (PCR) is used to further detection of IBDV, and the size of virus is confirmed with electron microscopy after sucrose gradient. Many methods are known to be used for the determination of anti-virus antibody titers, for example immunofluorescence (IF), virus neutralization (VN), enzyme-linked immunosorbent assay (ELISA), agar gel diffusion precipitation (AGP) and the like.

The virus neutralization (see, for example, Jackwood et al., Avian dis. 1986, 31, 370-375; Thayer et al., Avian Dis. 1986, 31, 120-124; and Jorge et al., Clin. Diagn. Lab. Immunol. 2000, 7, 645-651) is a reliable method, though it is limilted by the variance of embryonic eggs obtained in different seasons, and the time-spent production of chicken embryo fibroblasts (CEF) cell culture. The agar gel diffusion precipitation (see, for example, Box, P., in Proceedings of the 37^{th} Western Poultry Disease Conference, Davis, Calif., 1988, 21-24; and Rosenberger et al., in Isolation and Identification of Avian Pathogens, 3^{rd} ed., Kendall/Hunt Press, Dubuque, Iowa, 1989, 165-166) requires the preparation of large amount of antigen and positive serum samples, and has a further disadvantage that it may make diagnostic mistakes for the presence of non-specific precipitation lines. The disadvantages of immunofluorescence (see, for example, Allen et al., Avian Pathol. 1984, 13, 419-427; and Kumar etal, Indian Vet. J. 1993, 15, 26-29) include contamination of bacteria and fluorescence decay in observation period, which may affect the accuracy of results.

Alternatively, PCR has been widely used in diagnostic of diseases in veterinary (see, for example, Lee et al., J Clin Microbiol. 1994, 32, 1268-1272; and Mittal et al., International Journal of Poultry Science. 2005, 4, 239-243). PCR is a highly sensitive and specific detection method of IBD, though its operation should be very careful for that the PCR samples maybe contaminate with each other. The PCR is usually inhibited by certain substance such as phenol, SDS, heparin, bile acid, Dnase or Proteinase K and the like, though some of which may be removed by heating (see, for example, Schunck et al., J. Virol. Methods. 1995, 55, 427-433).

The IBDV genome consists of two segments of double-stranded RNA, designated as A and B of size 3.0 and 3.4 Kb, respectively. Segment A encodes a 108-kDa polypeptide that is self-cleaved to produce VPX (48 kDa), VP3 (32 kDa), and VP4 (28 kDa). In the mature virions, VPX is processed into VP2 (41 kDa). VP3 is a structural group-specific protein of the IBDV and has been suggested to be the major immunogenic protein of IBDV, since the earliest antibodies that appear after infection with live or inactivated viruses are directed to it .

As described above, detection of antibodies to IBDV has been accomplished by a number of different assays, and ELISA is the most often used method because it is economical and can quickly test large numbers of samples (Marquardt et al., Avian Dis. 1980, 24, 375-385). Commercial ELISA kits are presently available to detect antibodies for IBDV in field samples. The antigen source for these kits are based on the whole virion (see, for example, Marquardt etal, supra; Snyder et al., Avian Dis. 1984, 28, 12-24; Briggs et al., Avian Dis. 1986, 30, 216-218; Snyder et al., Avian Dis. 1986, 30, 139-148; and Thayer et al., supra), which is usually purified through a cesium chloride gradient by ultracentrifugation. In 1996, Jackwood (jackwood et al., supra) used IBDV recombinant protein to detect IBDV-specific antibody, and found that VP2 could induce neutralized antibody immune response to protect chicken from IBDV infection (see Jackwood et al., Avian Dis. 1999, 43, 189-197). In 2000, Jorge et al. (Jorge et al., supra) reported that the sensitivity and specificity of IBDV-specific detection using pVP2 expressed in insect cells than those of using VP3 protein. Meanwhile, the commercial IBD-detecting kit, such as which provided by IDEXX, KPL corp., uses isolated IBDV particles as antigen of ELISA, but it leads to high cost and time consumption.

However, related antigens expressed in E. coli host system haven't been used as antigen of IBDV-specific ELISA. The present invention therefore used recombinant VP2H and/or VP3 proteins expressed in insect cells and E. coli host system, replacing IBDV particles, as the antigen of indirect-ELISA to lower costs and improve efficiency of IBDV detection.

Recombinant VP3 protein has been produced in E. coli host system (see, Cheng, Y.-H., Master thesis, Graduate Institute of Biotechnology, National Chung Hsing University, Taichung (2003)). Among the recently developed purification techniques, immobilized metal affinity chromatography (IMAC) is a promising technique for the purification of recombinant histidine-tagged proteins due to its advantages of high recovery, high capacity, and complete regeneration (see, for example, G. P. Vladka and M. Viktor, J. Biochem. Biophys. Methods. 49 (2001) 335-7; G. S. J. Chaga, J. Biochem. Biophys. Methods. 49 (2001) 313; and E. K. M. Ueda et al., J. Chromatogr. 988 (2003) 1). Immobilized metal ion affinity method is based on the affinities between certain metal-binding amino acids in the side chains of proteins (e.g. histidine, cysteine, tyrosine, etc.), and the metal ions (e.g. Cu²⁺, Ni²⁺, Zn²⁺, etc.) chelated by the chelating agents (e.g. iminodiacetic acid (IDA), nitrilotriacetic acid (NTA), etc.) immobilized on the solid supports.

According to the present invention, to further explore the application of the pure VP3 proteins produced using the expression method, one recently developed immobilized metal affinity membrane (IMAM) process, instead of using the conventional packed-column chromatographic process, is use in the VP3 purification for achieving better mass-transfer performance. The membrane system has the advantages such as negligible diffusion limitation, lower pressure drop, higher accessible flow rate, simpler design, easier scale up, and so on (see, for example, D. K. Roper & E. N. Lightfoot, J. Chromatogr. A 702 (1995) 3; C.-Y. Wu et al., J. Chromatogr. 996 (2003) 53; and Y.-C. Liu et al., J. Chromatogr. 794 (2003) 67). Therefore, the IMAMs have been successfully applied in isolating or purifying enzymes, albumins, immunoglobulins, hemoglobins, ribonucleases, growth factors, histidine-tagged viruses, etc (see, for example, S.-Y. Suen, et al., J. Chromatogr. B 797 (2003) 305; and Y.-C. Liu, et al., J. Membr. Sci. 251 (2005) 201). Addition to the use of RC-based IMAM process, optimal conditions, such as loading buffer, elution buffer and flow rates, for recombinant VP3 purification have also been established in the present invention. And the purified VP3 recombinant proteins have been employed to develop a detection kit for IBDV identification. The development is successful and the cost of the kit is relatively low. The related results will be illustrated in the description below.

### SUMMARY OF THE INVENTION

The object of the present invention is to provide an ELISA kit for diagnosing infectious bursal disease (IBD), which is characterized by comprising recombinant VP2H or VP3 protein as the antigen for detecting specific anti-IBDV. In one embodiment of the invention, the recombinant VP2H or VP3 protein was produced in E. coli expression system.

Another object of the present invention is to provide an indirect-ELISA method for diagnosing infectious bursal disease (IBD), which comprising:

(a) coating ELISA plate wells with 0.1 μg to 0.5 μg/100 μpl/well of recombinant VP2H or VP3 protein;

(b) blocking with 5% skim milk in PBS;

(c) adding PBS-T-diluted (in 500 to 1000× dilution) serum sample from tested chicken to each well;

(d) washing with PBS-T buffer;

(e) adding PBS-T-diluted enzyme-conjugated secondary antibody;

(f) washing with PBS-T buffer;

(g) adding a developing reagent; and

(h) reading absorbance at suitable wavelength.

In one embodiment, the secondary antibody is HRP-conjugated anti-chicken antibody, the developing reagent is o-phenylenediamine dihydrochloride (OPD), the development reaction is stopped with 3 M HCl solution, and the absorbance is read at OD₄₉₀.

A further object of the present invention is to provide a purifying process for improving purity and recovery of recombinant VP3 protein, which is characterized by: using a Ni²⁺-IDA regenerated cellulose-based membrane, wherein the loading buffer comprising 20 mM NaH₂PO₄, 500 mM NaCl, and 10 mM imidazole, pH 6.5; the elution buffer comprising 20 mM NaH₂PO₄, 500 mM NaCl, and 500-750 mM imidazole, pH 7.8; with the loading flow rate of 1.7 ml/min and elution flow rate of 2.7 ml/min.

### BRIEF DESCRIPTION OF THE DRAWINGS

FIG. 1. shows the purification of TVP3 and ΔTVP3 proteins using IMAC, in which Lane 1: VP3 protein in the supernatant of BL21 (DE3) plysS cell lysate; Lane 2: VP3 protein in the crude extract after binding of the supernatant of BL21 (DE3) plysS cell lysate to Ni²⁺-NTA resin; Lane 3: eluted with Washing buffer; and Lane 4: purified VP3 protein.

FIG. 2. describes the detection of anti-VP3 Ab with indirect-ELISA, and the determination of optimal concentration of recombinant VP2H or VP3 protein for detecting IBDV-specific antibody. The used antigen is IMAC-purified TVP3 protein (A), ΔTVP3 protein (B) and VP2H protein (C), at concentration of 0, 0.1, 0.2, 0.3, 0.4 and 0.5 μg/100 μl/well. The primary antibody (in 1/500× dilution) is (▪) negative control, uninfected chicken (A3-8-6), (♦) positive control, infected chicken (B3-10-25), (□) positive control, infected chicken (B4-10-26) and (▾) Mock, Specific pathogen free (SPF) chicken. The secondary antibody (in 1/1000× dilution) is HRP-conjugated rabbit anti-chicken Ab.

FIG. 3. shows the correlation between VP2H- or ΔTVP3-ELISA and commercial ELISA kit. The serum is used in 1/500× dilution to detect the Ab titers and OD value determined with commercial ELISA kit and VP2H- or VP3-ELISA.

### DETAILED DESCRIPTION OF THE INVENTION

The present invention is further illustrated by the following examples, which, however, are not to be construed as limiting the scope of protection. The features disclosed in the foregoing description and in the following examples may, both separately and in any combination thereof, be material for realizing the invention in diverse forms thereof.

### EXAMPLE 1.

Expression and Preparation of Crude TVP3 and ΔTVP3 Extract

The cells of E. coli BL21 (DE3) plysS, which contained a recombinant pET28a plasmid carrying TVP3 or ΔTVP3 gene, were grown at 37° C. at 225 rpm shaking overnight. 1 ml of the overnight culture was inoculated to 100 ml of fresh Luria-Bertani medium in the presence of 50 μg/ml Kanamycin and grown to OD₆₀₀ of 0.5˜0.6 at 37° C. The protein expression was induced by adding IPTG at a final concentration of 1 mM. After 3 h, the cells were pelleted at 10,000 g (Kubota Ra 200-J rotor, Tokyo, Japan) and at 4° C. for 15 min, and then lysed on ice by an ultrasound in a lysis buffer (20 mM NaH₂PO₄, 500 mM NaCl, 10 mM imidazole, pH=6.5˜6.7) for 2 sec at interval of 1 sec, total 10 min. Cell lysates were centrifuged at 10,000 g and 4° C. for 15 min, and the supernatant was collected and stored at −20° C. for further purification.

The cell lysate supernatant was loaded to a column containing about 20 mL Ni²⁺-NTA resin. After 1 hr of adsorption, the protein was eluted with imidazole gradient. At the first, the column was washed with 10× volume of lysis buffer to remove the proteins that didn't bind to Ni²⁺, then washed with 10× volume of washing buffer (20 mM NaH₂PO₄, 500 mM NaCl, 40 mM imidazole, pH=7.8˜8.0) to remove the non-specific binding proteins, and finally using 2× volume of elution buffer (20 mM NaH₂PO₄, 500 mM NaCl, 500 mM imidazole, pH=7.8˜8.0) to elute the object proteins.

By the results of SDS-PAGE and Western blotting, it was showed that molecular weight of the E. coli-expressed VP3 protein (TVP3) and its C-terminally truncated ΔTVP3 is about 32 and 26 kDa, respectively.

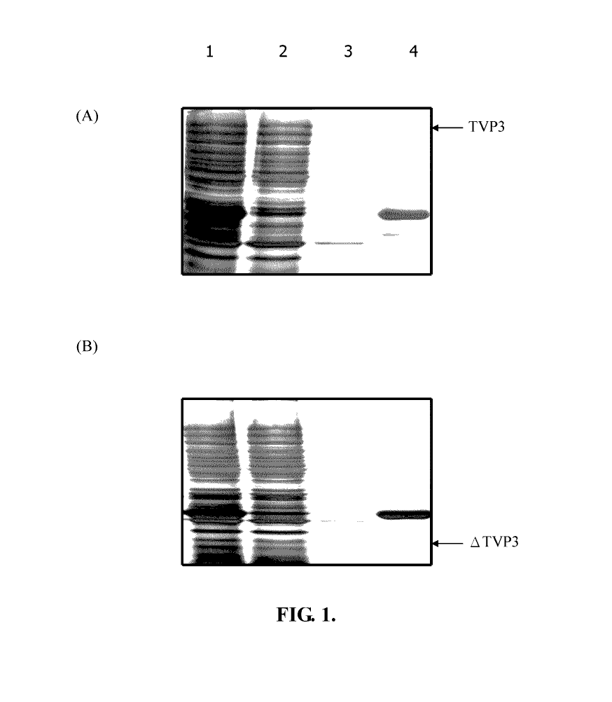

In order to obtain higher recovery yield, the procedure described by Cheng (2003, supra) was modified that the pH of lysis buffer was decreased to 6.5˜6.7; and the concentration of imidazole in washing buffer was changed to 40 mM; using 500 mM imidazole could elute TVP3 or ΔTVP3 completely. According to the purification conditions described above, could prepare object proteins TVP3 (FIG. 1A., Lane 4) and ΔTVP3 (FIG. 1B., Lane 4) with high purity (98˜99%) and raised yield of 86˜88% (FIG. 1 and Table 2). In such conditions, we could produce 140˜150 mg of P3protein from 1 Kg bacteria, and obtain 139˜147 mg of protein after purification with recovery yield of about 98%˜99%.

### EXAMPLE 2

Eenzyme-Linked Immunosorbent Assay (ELISA) with Recombinant VP2 or VP3

In the experiment, IBDV Ab-positive antiserum was obtained from vvIBDV (virulent IBDV strain) infected chicken, named as B3-10-25 and B3-10-26. VP2 Ab-positive antiserum and VP3 Ab-negative antiserum was obtained from the IBDV uninfected chicken by immunizing with VP2 protein, which serum named as A3-8-6. The IBDV Ab-negative serum was obtained from Specific pathogen free (SPF) chicken, which serum named as Mock. The 296 serum samples of farmed chicken were provided by Dr. Shen's lab. (Department of Veterinary Medicine, National Chung Hsing University, Taichung).

For the preparation of indirect-ELISA, VP2H-ELISA and VP3-ELISA, 96-well plates (EIA/RIA strip plate, Costar, Cambridge, Mass., USA) were coated with 100 μl of recombinant VP2H or VP3 at concentration of 0 μg, 0.1 μg, 0.2 μg, 0.3 μg, 0.4 μg and 0.5 μg/ml, diluted with coating buffer (0.14 M NaCl, 1 M Na₂CO₃, 1 M NaCO₃, pH=9.6). After coating at 4° C. for 4 hr, blocking procedure was carried out by incubating with 200 μl of blocking reagent (5% skim milk in PBS) at 4° C. overnight. After washing with PBST three times, 500× diluted serum sample was added and the plates were incubated at 4° C. for 2 hr, then washed with PBST buffer three times, each for 10 min. The plates were treated with horseradish peroxidase (HRP)-conjugated goat anti-rabbit IgG (Jackson Immuno Research Laboratories, West Grove, Pa., USA) diluted to 1:1000 in PBST buffer for 2 h at 4° C. Following similar washes, 100 μl of o-phenylenediamine dihydrochloride (OPD) substrate solution (Sigma) was added to each well, and the plates were incubated for 7 min for color development. After adding 50 μl of 3 M HCl to stop reaction, the absorbance values were measured at 490 nm with an ELISA reader (Dynex Technologies, Chantilly, Va., USA). Additionally, the positive cutoff value obtained from 30 sets of SPF chicken serum is of about OD=0.12, which is used as criterion of positive sample in the field tests.

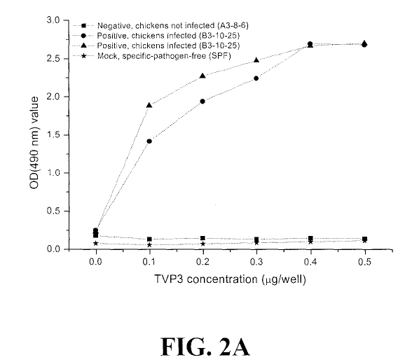

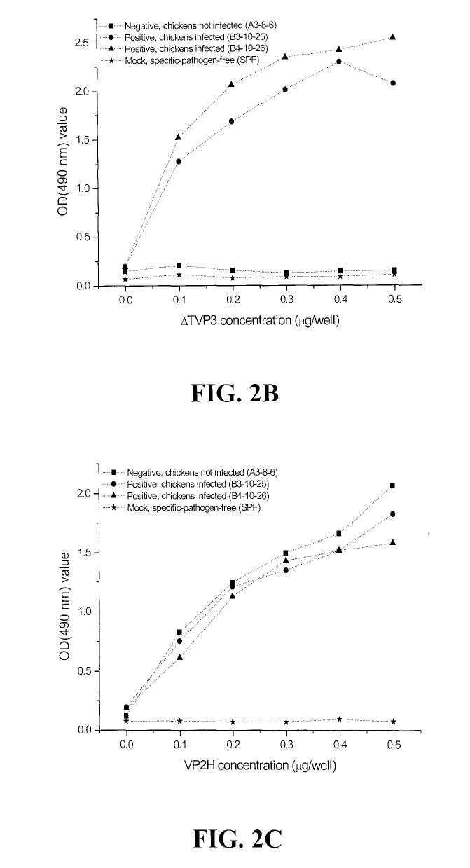

From the results, the VP3 Ab-specific signal was actually detected in B3-10-25 and B3-10-26 serum by the VP3-ELISA (see, FIGS. 2A and 2B). In the contrast, it showed no VP3 Ab-specific signal detected in A3-8-6 and Mock serum by the same indirect VP3-ELISA (FIGS. 2A and 2B). Therefore, it is strongly suggested that the recombinant VP3 purified according to the present process could specifically detect anti-IBDV antibody in the serum of infected chicken.

Furthermore, it was shown in FIG. 2 that IBDV-specific antibodies were detected at 500× dilution of chicken sample serum and in 0.1 μg/100 μl/well of VP2H and VP3 protein. Thus, we chose the VP2H or VP3 protein of 0.1 μg/100 μl/well to coat on ELISA plates for preparing ELISA kits.

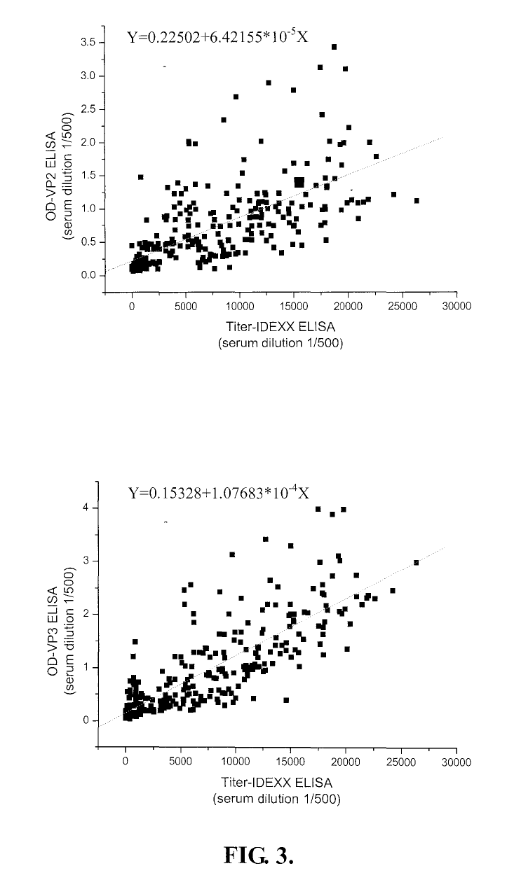

To evaluate the utility of recombinant VP2H or ΔVP3 as the antigen in ELISA detection kit, we collected 292 sets of field chicken serum to detect the presence of anti-IBDV antibody with the indirect-ELISA kits, and further compared the OD values and Ab titers obtained by the present VP2H-ELISA or VP3-ELISA kit with those by commercial ELISA kit (IDEXX kit, which containing intact IBDV particles isolated by ultra-centrifugation). As shown in FIG. 3, three are 262 sets of positive serum and 30 sets of negative serum in the collected field chicken serum.

We also assayed the relativities of OD value and Ab titers. The present VP2H-ELISA and VP3-ELISA kits and commercial ELISA kit (IDEXX kit) were first used to detect anti-IBDV antibody in the field sample serum, then assay the correlation between the ELISA kits and serum neutralizing antibody tests (challenged with 200TCID₅₀ virulent strain). The results are listed in Tables 1-1 to 1-3.

TABLE 1-1
Evaluation of commercial ELISA kit and the correlation
with neutralizing antibody test.

| | SN^{c} | | | |
|---|---|---|---|---|
| IDEXX-ELISA^{a} | | Positive | Negative | Total |

| | | | | | |
|---|---|---|---|---|---|
| Positive | 257 | (98.5) | 27 | (93.1) | 284 |
| Negative | 4 | (1.5) | 2 | (6.9) | 6 |
| Total | 261 | | 29 | | 290 |

| | |
|---|---|
| Sensitivity | 98% |
| Specificity | 7% |
| Accuracy | 89% |
| Kappa | 0.89 |

^{a}For IDEXX kit, the judgment criterion to be IBDV positive or negative is calculated according to the instruction provided by manufacturer.
^{b}For VP2H- and VP3-ELISA kit, the sample showed an OD value more than the cut value (0.12) was considered as positive.
^{c}If serum exhibited antibody titer >32, then it was considered as positive. The percentage was calculated as the percentage of the serum number as positive or negative in ELISA related to the serum number as positive or negative in the neutralizing antibody test.

TABLE 1-2
Evaluation of VP2H-ELISA kit and the correlation
with neutralizing antibody test.

| | SN^{c} | | | |
|---|---|---|---|---|
| VP2H-ELISA^{b} | | Positive | Negative | Total |

| | | | | | |
|---|---|---|---|---|---|
| Positive | 251 | (96.2) | 4 | (13.8) | 255 |
| Negative | 10 | (3.8) | 25 | (86.2) | 35 |
| Total | 261 | | 29 | | 290 |

| | |
|---|---|
| Sensitivity | 96% |
| Specificity | 86% |
| Accuracy | 95% |
| Kappa | 0.95 |

^{a}For IDEXX kit, the judgment criterion to be IBDV positive or negative is calculated according to the instruction provided by manufacturer.
^{b}For VP2H- and VP3-ELISA kit, the sample showed an OD value more than the cut value (0.12) was considered as positive.
^{c}If serum exhibited antibody titer >32, then it was considered as positive. The percentage was calculated as the percentage of the serum number as positive or negative in ELISA related to the serum number as positive or negative in the neutralizing antibody test.

TABLE 1-3
Evaluation of ΔTVP3-ELISA kit and the correlation
with neutralizing antibody test.

| | SN^{c} | | | |
|---|---|---|---|---|
| ΔTVP3-ELISA^{b} | | Positive | Negative | Total |

| | | | | | |
|---|---|---|---|---|---|
| Positive | 262 | (100) | 2 | (7.1) | 264 |
| Negative | 0 | (0) | 26 | (92.9) | 26 |
| Total | 262 | | 28 | | 290 |

| | |
|---|---|
| Sensitivity | 100% |
| Specificity | 93% |
| Accuracy | 99% |
| Kappa | 0.99 |

^{a}For IDEXX kit, the judgment criterion to be IBDV positive or negative is calculated according to the instruction provided by manufacturer.
^{b}For VP2H- and VP3-ELISA kit, the sample showed an OD value more than the cut value (0.12) was considered as positive.
^{c}If serum exhibited antibody titer >32, then it was considered as positive. The percentage was calculated as the percentage of the serum number as positive or negative in ELISA related to the serum number as positive or negative in the neutralizing antibody test.

As shown in Tables 1-1 to 1-3, in 292 sets of chicken serum, 262 sets exhibited positive results in neutralizing antibody test, 257 sets exhibited positive results in commercial ELISA test, and 251 and 262 sets exhibited positive results in VP2H- and VP3-ELISA tests, respectively. The similarity between results obtained from VP2H- or VP3-ELISA tests and neutralizing antibody test was 96.2% and 100%, respectively, and the similarity between the commercial ELISA kit and neutralizing antibody test was 98.5%. Accordingly, there are good correlations between the present ELISA kit and the neutralizing antibody test. Moreover, the reliability of the commercial ELISA kit was 0.89 (see Table 1-1), while the reliability in the present VP2H- or VP3-ELISA test was 0.95 (as shown in Table 1-2) or 0.99 (as shown in Table 1-3). It is significant that the present ELISA kit could be used in detecting IBDV-specific antibody in sample.

Furthermore, we compared the results described above with those published by Jorge (Jorge et al., 2000, supra), and found that the similarity of the present ΔTVP3-ELISA tests and neutralizing antibody test (100%) was comparable to the test using recombinant VPX protein expressed in insect cell system as antigen (100%), and it was better than the ELISA using antigen VP3 described in the prior art (96%).

### EXAMPLE 3

Purification of Recombinant VP3 Protein by Immobilized Metal Affinity Membrane (IMAM) Process

Preparation of Regenerated Cellulose-Based IMAM

All the reactions were carried out in a 120-ml glass bottle. A piece of RC membrane disc was incubated in a solution of 5 ml epichlorohydrin at aqueous phase (20 ml of 1 M NaOH) or at alcohol phase (5 ml of 1 M NaOH mixed with 15 ml of 98% ethanol) and shaken under a constant shaking rate at 60° C. for 2 h. After incubation, the membrane was rinsed with deionized (DI) water. Reaction conditions such as shaking rate and epichlorohydrin phase were varied to study their effects on the coupled epichlorohydrin density. For coupling IDA, the epichlorohydrin-conjugated membrane was reacted with 25 ml of 0.2 M IDA and 1 M Na₂CO₃, pH 11, at 80° C. for 12 h. After reaction, the membrane was washed with 5% acetic acid and DI water. Each modified membrane was immersed in 10 ml of 0.05 M NiSO₄ solution for 1 h, then rinsed with DI water in order to remove the unbound or weakly bound nickel ions.

Batch Purification Experiments

For batch experiments, 1 ml of Ni²⁺-NTA commercial agarose gel (10.21±0.38 μmol Ni²⁺/ml) or 4 pieces of Ni²⁺-IDA regenerated cellulose-based membrane (10.48±0.67 μmol Ni²⁺/4 disks) were put into a glass bottle and 10 ml of crude protein extract in the lysis buffer was loaded. The incubation was conducted at 4° C. for 1 h. After binding, the glass bottle was washed by 10 ml of loading buffer (lysis buffer) and 10 ml of washing buffer (20 mM NaH₂PO₄, 500 mM NaCl, 40 mM imidazole, pH 7.8). Then, the VP3 proteins were eluted using 2 ml of elution buffer (20 mM NaH₂PO₄, 500 mM NaCl, 500 mM or 750 mM imidazole, pH 7.8). Different imidazole concentrations in the elution buffer were tested in this work.

Both the Ni²⁺-NTA commercial agarose gels and Ni²⁺-IDA RC-based membranes were used for the batch purification of VP3 and some operation conditions were selected to achieve higher purification efficiency. 1 ml of Ni²⁺-NTA commercial agarose gel and 4 pieces of Ni²⁺-IDA regenerated cellulose-based membrane were employed because their Ni²⁺ capacities were very close.

The results from batch experiments are listed in Table 2. After loading the crude protein lysate onto the affinity column or membrane holder, a few proteins other than VP3 were also bound to the column or membranes as some of the native E. coli proteins have histidine at their molecular surface. In order to reduce the binding of these impurity proteins, the lysis buffer with 10 mM imidazole and pH 6.5 was adopted directly as the loading buffer and most of VP3 proteins were adsorbed (more than 98%). When applying this lysis buffer for washing, only few VP3 but some impurity proteins were washed out. When further applying the buffer with 40 mM imidazole and pH 7.8 for the second washing, a small amount of impurity proteins was removed.

The VP3 could be entirely eluted out with the use of 500 mM imidazole, pH 7.8 for the commercial gels, but the assistance of higher-concentration (750 mM) imidazole buffer was necessary for a clear VP3 elution from the IMAMs. This phenomenon could be explained as a result of the stronger binding between the tridentate IDA-chelated Ni²⁺ and the VP3 proteins in the membrane system, compared to that between the tetradentate NTA-chelated Ni²⁺ and the proteins in the gels. About 11˜12-fold purification and 95˜98% VP3 recovery were achieved by using both matrices in the batch process (Table 2).

TABLE 2
VP3 purification performance in the batch process.

| | Stage | | | | | | | | | |
|---|---|---|---|---|---|---|---|---|---|---|
| | ΔTVP3 | | | | | TVP3 | | | | |
| | | Total | | ΔTVP3 | | | Total | | TVP3 | |
| | Volume | protein | ΔTVP3 | recovery | Purification | Volume | protein | TVP3 | recovery | Purification |
| | (ml) | (mg) | (mg) | (%) | fold | (ml) | (mg) | (mg) | (%) | fold |
| Ni²⁺-NTA gels | | | | | | | | | | |
| Crude lysate | 10 | 19.13 | 1.51 | — | — | 10 | 17.41 | 1.46 | — | — |
| Residue | 10 | 16.43 | 0.02 | 0.99 | 0.01 | 10 | 13.90 | 0.02 | 1.44 | 0.02 |
| Wash (10 mM | 10 | 1.18 | 0.01 | 0.53 | 0.06 | 10 | 1.98 | 0.01 | 0.83 | 0.08 |
| imidazole; pH = 6.5) | | | | | | | | | | |
| Wash (40 mM | 10 | 0.03 | 0.02 | 0.19 | 3.62 | 10 | 0.12 | 0.03 | 2.34 | 3.36 |
| imidazole; pH = 7.8) | | | | | | | | | | |
| Elution (500 mM | | | | | | 10 | 1.41 | 1.39 | 95.40 | 11.78 |
| imidazole; pH = 7.8) | | | | | | | | | | |
| Elution (750 mM | | | | | | | | | | |
| imidazole; pH = 7.8) | | | | | | | | | | |
| Ni²⁺-IDA | 10 | 18.64 | 1.53 | — | — | 10 | 17.89 | 1.47 | — | — |
| membranes | | | | | | | | | | |
| Crude lysate | 10 | 15.94 | 0.01 | 0.33 | 0.01 | 10 | 14.41 | 0.01 | 0.68 | 0.01 |
| Residue | 10 | 1.15 | 0.00 | 0.08 | 0.01 | 10 | 1.99 | 0.01 | 0.54 | 0.05 |
| Wash (10 mM | 10 | 0.01 | 0.00 | 0.20 | 0.04 | 10 | 0.04 | 0.00 | 0.28 | 1.28 |
| imidazole; pH = 6.5) | | | | | | | | | | |
| Wash (40 mM | 10 | 1.00 | 0.99 | 64.86 | 12.07 | 10 | 1.03 | 0.98 | 66.78 | 11.61 |
| imidazole pH = 7.8) | | | | | | | | | | |
| Elution (500 mM | 10 | 0.54 | 0.54 | 35.14 | 12.18 | 10 | 0.48 | 0.47 | 32.07 | 12.08 |
| imidazole; pH = 7.8) | | | | | | | | | | |
| Elution (750 mM | | | | | | | | | | |
| imidazole pH = 7.8) | | | | | | | | | | |

Flow Purification Experiments

For flow experiments at 4° C., 1 ml of Ni²⁺-NTA commercial agarose gel or 4 pieces of Ni²⁺-IDA regenerated cellulose-based membrane (under the optimal preparation conditions) were employed. The gels were packed as a slurry into an acrylic column (1.0-cm diameter) to give a 1.9 cm bed, and the membrane chromatographic system was comprised of an acrylic 47 mm-membrane cartridge (lab made). 10-ml of crude VP3 extract was loaded at a constant flow rate using a peristaltic pump (MP-3N, Eyela, Tokyo, Japan) to the column or cartridge already equilibrated with the lysis buffer. Unbound proteins were then washed out with 10 ml of washing buffer at 2.7 ml/min. Finally, bound proteins were eluted with 2 ml of elution buffer at a constant flow rate.

The purification performance of VP3 using the affinity gel column or membrane cartridge at different flow rates is shown in Table 3. Proper binding of the target protein to the immobilized metal ions requires sufficient time to attain equilibrium. At comparatively higher flow rates (such as 2.3 and 3 ml/min), the loaded protein did not get enough time for binding, and therefore the recovery of VP3 was low (62˜65%). When the flow rate was decreased, the VP3 recovery increased. The optimum flow rate was found to be 1.7 ml/min for protein loading and 2.7 ml/min for protein washing or elution, because the increase in recovery for further lower flow rates was not significant.

Moreover, both matrices obtained similar recovery and purity results as shown in Table 3. Regarding that the IMAM process could offer simpler design, shorter process time (less than 12% of the process time for IMAC), and lower pressure drop, its process efficiency in a larger-scale system should be superior to the gel bead column. Therefore, VP3 purification with IMAMs is a potential approach in industry.

TABLE 3
VP3 purification performance in the flow process at different flow rates.

| | | ΔTVP3 | | | | | | TVP3 | | | | | |
|---|---|---|---|---|---|---|---|---|---|---|---|---|---|
| | | Ni⁺²-NTA gels | | | Ni⁺²-IDA membranes | | | Ni⁺²-NTA gels | | | Ni⁺²-IDA membranes | | |
| | | Total | | | Total | | | Total | | | Total | | |
| Flow rate | | time | Recovery | Purity | time | Recovery | Purity | time | Recovery | Purity | time | Recovery | Purity |
| (ml/min) | | (min) | (%) | (%) | (min) | (%) | (%) | (min) | (%) | (%) | (min) | (%) | (%) |
| (1) | Loading 0.5 | 58.0 | 93.6 | 100.0 | 50.0 | 91.3 | 100.0 | 57.7 | 92.1 | 100.0 | 49.2 | 90.4 | 100.0 |
| | Washing 1.0 | | | | | | | | | | | | |
| | Elution 1.0 | | | | | | | | | | | | |
| (2) | Loading 1.0 | 32.4 | 90.2 | 99.6 | 27.6 | 89.7 | 99.3 | 31.9 | 89.7 | 99.9 | 26.4 | 88.8 | 99.1 |
| | Washing 1.7 | | | | | | | | | | | | |
| | Elution 1.7 | | | | | | | | | | | | |
| (3) | Loading 1.7 | 20.2 | 88.5 | 99.0 | 17.9 | 87.1 | 98.7 | 19.4 | 87.4 | 99.3 | 17.0 | 86.5 | 98.4 |
| | Washing 2.7 | | | | | | | | | | | | |
| | Elution 2.7 | | | | | | | | | | | | |
| (4) | Loading 2.3 | 17.6 | 64.6 | 98.2 | 15.9 | 62.4 | 98.0 | 16.7 | 63.5 | 98.4 | 15.1 | 62.0 | 98.2 |
| | Washing 3.0 | | | | | | | | | | | | |
| | Elution 3.0 | | | | | | | | | | | | |

Protein loading: 10 ml, 1.51 mg/ml.
Loading buffer: 20 mM NaH₂PO₄, 500 mM NaCl, 10 mM imidazole, pH 6.5.
Washing buffer: 20 mM NaH₂PO₄, 500 mM NaCl, 40 mM imidazole, pH 7.8.
Elution buffer: 20 mM NaH₂PO₄, 500 mM NaCl, 500 mM imidazole for gels or 750 mM imidazole for membranes, pH 7.8.
All experiments were repeated twice.

What is claimed is:

1. An ELISA kit for diagnosing infectious bursal disease (IBD), comprising recombinant VP2H or VP3 protein as the antigen for detecting specific anti-IBDV, in which the recombinant VP2H or VP3 protein is produced in E. coli expression system.

2. The ELISA kit of claim 1, wherein the E. coli expression system is BL21 (DE3) plysS.

3. The ELISA kit of claim 1, wherein the recombinant VP3 protein is an intact VP3 protein.

4. The ELISA kit of claim 1, wherein the recombinant VP3 protein is a C-terminally truncated VP3 protein.

5. An indirect-ELISA method for diagnosing infectious bursal disease (IBD), comprising:
(a) coating ELISA plate wells with recombinant VP2H or VP3 protein;
(b) blocking with 5% skim milk in PBS;
(c) adding PBS-T-diluted serum sample from tested chicken to each well;
(d) washing with PBS-T buffer;
(e) adding PBS-T-diluted enzyme-conjugated secondary antibody;
(f) washing with PBS-T buffer;
(g) adding a developing reagent; and
(h) reading absorbance at suitable wavelength.

6. The indirect-ELISA method of claim 5, wherein the recombinant VP2H or VP3 protein is coated at the concentration of 0.1 μg to 0.5 μg/100 μl/well.

7. The indirect-ELISA method of claim 5, wherein the PBS-T-diluted serum sample from tested chicken is diluted to 1/500 to 1/1000.

8. The indirect-ELISA method of claim 5, wherein the secondary antibody is HRP-conjugated anti-chicken antibody, the developing reagent is OPD (o-phenylenediamine dihydrochloride) and stopped with 3 M HCl and detected at OD₄₉₀.

9. A purifying process for improving purity and recovery of recombinant VP3 protein, which is characterized by: using a Ni²⁺-IDA regenerated cellulose-based membrane, wherein the loading buffer comprising 20 mM NaH₂PO₄, 500 mM NaCl, and 10 mM imidazole, pH 6.5; the elution buffer comprising 20 mM NaH₂PO₄, 500 mM NaCl, and 500-750 mM imidazole, pH 7.8; with the loading flow rate of 1.7 ml/min and elution flow rate of 2.7 ml/min.

10. The process of claim 9, wherein the recombinant VP2H or VP3 protein is produced in E. coli expression system.

11. The process of claim 9, wherein the recombinant VP3 protein is an intact VP3 protein.

12. The process of claim 9, wherein the recombinant VP3 protein is a C-terminally truncated VP3 protein.
